(19) United States
(12) Patent Application Publication
(10) Pub. No.: US 2025/0387627 A1
(43) Pub. Date: Dec. 25, 2025

(54) STIMULATION OPTIMIZATION BASED ON BRAIN WAVES

(71) Applicant: Boston Scientific Neuromodulation Corporation, Valencia, CA (US)

(72) Inventor: Leon Mauricio Juarez Paz, Berlin (DE)

(21) Appl. No.: 19/237,752

(22) Filed: Jun. 13, 2025

Related U.S. Application Data
(60) Provisional application No. 63/662,265, filed on Jun. 20, 2024.

(51) Int. Cl. A61N 1/36; A61N 1/372; G16H 20/40; G16H 40/63

(52) U.S. Cl. CPC A61N 1/36139; A61N 1/36132; A61N 1/3615; A61N 1/37247; G16H 20/40; G16H 40/63

(57) ABSTRACT

This document discusses a programming system for a neurostimulation device that provides neurostimulation energy to a patient according to programmable stimulation parameters. The programming system includes a wearable brain wave signal sensing device configured to sense brain wave signals of a patient produced in response to neurostimulation delivered using the neurostimulation device, and a second device. The second device includes a communication circuit to receive brain wave signal information from the wearable brain wave signal sensing device, processing circuitry, and a client application. The client application includes instructions executable by the processing circuitry and is configured to determine a sensed response to the neurostimulation of the patient indicated by the received brain wave signal information, compare the sensed response to a desired target response to the neurostimulation, and adjust the stimulation parameters of the neurostimulation device according to the comparing of the sensed response and the desired target response.

### CLAIM OF PRIORITY

This application claims the benefit of U.S. Provisional Application No. 63/662,265, filed on Jun. 20, 2024, which is hereby incorporated by reference in its entirety.

### TECHNICAL FIELD

This document relates generally to medical devices, and more particularly, to systems, devices, and methods for determining and setting of stimulation parameters for programming an electrical neurostimulation system.

### BACKGROUND

Neurostimulation, also referred to as neuromodulation, has been proposed as a therapy for a number of conditions. Examples of neurostimulation include Deep Brain Stimulation (DBS), Spinal Cord Stimulation (SCS), Peripheral Nerve Stimulation (PNS), and Functional Electrical Stimulation (FES). Implantable neurostimulation systems have been applied to deliver such a therapy. An implantable neurostimulation system may include an implantable neurostimulator, also referred to as an implantable pulse generator (IPG), and one or more implantable leads each including one or more electrodes. The implantable neurostimulator delivers neurostimulation energy through one or more electrodes placed on or near a target site in the nervous system. An external programming device can be used to program the implantable neurostimulator with stimulation parameters controlling the delivery of the neurostimulation energy.

In one example, the neurostimulation energy is delivered in the form of electrical neurostimulation pulses. The delivery is controlled using stimulation parameters that specify spatial (where to stimulate), temporal (when to stimulate), and informational (patterns of pulses directing the nervous system to respond as desired) aspects of a pattern of neurostimulation pulses. Neurostimulation systems may offer many programmable options for the parameters of the neurostimulation to customize the neurostimulation therapy for a specific patient. For some types of neurostimulation (e.g., DBS) the efficacy of the neurostimulation for the patient may depend on an intricate balance of stimulation location coupled with the programmed stimulation waveform. Optimization of stimulation settings may be based on activity of brain neurons. This would involve sensing neuron activity (e.g., sensing local field potentials) using implanted stimulation leads while changing parameters of the stimulation delivered using the leads. However, using the implanted leads to sense signals usable as feedback for neurostimulation involves inherent challenges, such as low signal to noise ratio, signal artifacts from heart activity and muscle movement, the energy demand on the pulse generator to continuously sense and record the signals, ambiguity in the electrode recording the brain activity, inability to use certain stimulation features when sensing (e.g., a bipolar stimulation mode) and others.

### SUMMARY

In DBS, electrical neurostimulation therapy is delivered to implantable electrodes located at certain neurostimulation targets in the brain to treat neurological or neurophysiological disorders. Optimizing neurostimulation parameters to a particular patient using brain activity sensed with the same implanted leads used to provide the stimulation is challenging. Sensing brain activity using a wearable device when optimizing neurostimulation reduces the challenges in customizing neurostimulation for the patient.

A first Example (Example 1) includes subject matter such as a (of controlling operation of a neurostimulation system) including delivering the neurostimulation with first stimulation parameters using an implantable stimulation lead that includes electrodes, sensing response brain wave signals to the neurostimulation using a wearable brain wave signal sensing device, sending recorded response brain wave signals to a separate device, and determining, using the separate device, a response of the patient to the neurostimulation indicated by the recorded response brain wave signals, and recurrently adjusting the stimulation parameters to adjust the response to the neurostimulation indicated by the recorded response brain waves.

In Example 2, the subject matter of Example 1 optionally includes sending first brain wave signals from the wearable brain wave signal sensing device corresponding to an off-therapy state of the patient; sending second brain wave signals from the wearable brain wave signal sensing device corresponding to an on-therapy state of the patient; inputting the first and second brain wave signals to a machine learning algorithm of the separate device; and determining, using the machine learning algorithm, whether subsequent brain wave signals sent from the wearable brain wave signal sensing device are indicative of the response to the neurostimulation being the off-therapy state of the patient or the on-therapy state of the patient.

In Example 3, the subject matter of one or both of Examples 1 and 2 optionally includes sensing the brain wave signals using an electroencephalogram (EEG) headband; and sending the recorded response brain waves to a personal device of the patient that performs signal processing on the response brain waves to determine whether the response brain waves correspond to a desired target response to the neurostimulation.

In Example 4, the subject matter of one or any combination of Examples 1-3 optionally includes recurrently changing the stimulation parameters using the personal device of the patient.

In Example 5, the subject matter of Example 4 optionally includes delivering the neurostimulation using an implantable pulse generator, and the personal device of the patient is a smartphone that includes an application to change the stimulation parameters.

In Example 6, the subject matter of one or both of Examples 1 and 2 optionally includes sensing the brain waves using an electroencephalogram (EEG) headband and sending the recorded response brain waves to a cloud device that performs signal processing on the response brain waves to determine whether the response brain waves correspond to a desired target response to the neurostimulation.

In Example 7, the subject matter of one or any combination of Examples 1-6 optionally includes delivering the neurostimulation with a first stimulation energy amplitude and a first electrode fractionalization of the stimulation lead, determining whether the recorded response brain wave signals indicate that the first stimulation parameters produce a desired target response to the neurostimulation energy, and changing one or both of the electrode fractionalization and the stimulation energy amplitude when the recorded response brain wave signals indicate that the first stimulation parameters did not produce the desired target response.

In Example 8, the subject matter of one or any combination of Examples 1-7 optionally includes comparing, by the separate device, the recorded response brain wave signals to a predetermined sequence of brain wave signals; and recurrently adjusting the stimulation parameters to reduce a difference between the sensed response brain wave signals and the predetermined sequence of brain wave signals.

In Example 9, the subject matter of one or any combination of Examples 1-8 optionally includes comparing, by the separate device, the recorded response brain wave signals to a target response brain wave signal; and adjusting the stimulation parameters to change the sensed response brain wave signals to reduce a difference between the sensed response brain wave signals and the target response brain wave signal.

In Example 10, the subject matter of one or any combination of Examples 1-9 optionally includes sensing the brain wave signals using an electroencephalogram (EEG) headband; determining, using the separate device, a frequency response of the sensed brain wave signals sensed using the EEG headband; comparing, by the separate device, the determined frequency response to a target frequency response; and adjusting the stimulation parameters to change the frequency response of the sensed brain wave signals to reduce a difference between the frequency response of the recorded signals and the target frequency response.

Example 11 includes subject matter (such as programming system for a neurostimulation device that provides neurostimulation energy to a patient according to programmable stimulation parameters) or can optionally be combined with one or any combination of Examples 1-10 to include such subject matter, comprising a wearable brain wave signal sensing device configured to sense brain wave signals of a patient produced in response to neurostimulation delivered using the neurostimulation device, and a second device. The second device includes a communication circuit to receive brain wave signal information from the wearable brain wave signal sensing device, processing circuitry, and a client application including instructions executable by the processing circuitry. The client application is configured to determine a sensed response to the neurostimulation of the patient indicated by the received brain wave signal information; compare the sensed response to a desired target response to the neurostimulation; and adjust the stimulation parameters of the neurostimulation device according to the comparing of the sensed response and the desired target response.

In Example 12, the subject matter of Example 11 optionally includes a wearable brain wave sensing device that includes an electroencephalogram (EEG) headband, and the second device is a smartphone. The client application is optionally configured to receive the sensed brain wave signals produced by the EEG headband; perform signal processing on the sensed brain wave signals to determine whether the sensed brain wave signals correspond to the desired target response to the neurostimulation; and program the neurostimulation device with the adjusted stimulation parameters.

In Example 13, the subject matter of Example 12 optionally includes the client application is optionally configured to receive information from a user of the smartphone regarding the neurostimulation delivered using the neurostimulation device; and determine the sensed response of the patient to the neurostimulation using the sensed brain wave signals and the information from the user.

In Example 14, the subject matter of Example 11 optionally includes a wearable brain wave sensing device that includes an electroencephalogram (EEG) headband, and the wearable brain wave sensing device is configured to send the sensed brain wave signals to a personal device of the patient. The second device is a cloud device and the client application is configured to receive the sensed brain wave signals from the personal device; perform signal processing on the sensed brain wave signals to determine the sensed response to the neurostimulation indicated by the sensed brain waves and compare the sensed response to the desired target response to the neurostimulation; and send the adjusted stimulation parameters to the personal device.

In Example 15, the subject matter of Example 11 optionally includes a wearable brain wave sensing device that includes an electroencephalogram (EEG) headband, and the client application is optimally configured to compare the sensed brain wave signals sensed with the EEG headband to a target response brain wave signal, and adjust the stimulation parameters of the neurostimulation device to change the sensed brain wave signals to reduce a difference between the target response brain wave signal and the sensed brain wave signals sensed with the EEG headband.

In Example 16, the subject matter of one or any combination of Examples 11-15 optionally includes a client application configured to set the stimulation parameters of the neurostimulation device to include a first stimulation energy amplitude and a first electrode fractionalization; determine whether the sensed brain wave signals produced by the wearable brain wave sensing device indicate that the first stimulation energy amplitude and a first electrode fractionalization produce the desired target response to the neurostimulation; and change one or both of the electrode fractionalization and the stimulation energy amplitude when the sensed brain wave signals indicate that the first stimulation parameters did not produce the desired target response.

In Example 17, the subject matter of one or any combination of Examples 11-16 optionally includes a client application configured to determine a frequency response of the sensed brain wave signals sensed using the wearable brain wave sensing device; compare the determined frequency response to a target frequency response; and adjust the stimulation parameters to change the frequency response of the sensed brain wave signals to reduce a difference between the determined frequency and the target frequency response.

Example 18 includes subject matter (such as a monitoring system) or can optionally be combined with one or any combination of Examples 1-17 to include such subject matter, comprising a wearable brain wave sensing device configured to sense brain wave signals of a patient and a second device including a client application. The client application is configured to receive first brain wave signals from the wearable brain wave sensing device corresponding to an off-therapy state of the patient, receive second brain wave signals from the wearable brain wave sensing device corresponding to an on-therapy state of the patient, input the first and second brain wave signals to a machine learning algorithm, and determine, using the machine learning algorithm, whether subsequent brain wave signals received from the wearable brain wave sensing device are indicative of the off-medication state of the patient or the on-therapy state of the patient.

In Example 19, the subject matter of Example 18 optionally includes a wearable brain wave sensing device includes an electroencephalogram (EEG) headband and the second device being a smartphone. The client application is performable by processing circuitry of the smartphone and is optionally configured to present a prompt on a display of the smartphone regarding a determined therapy state of the patient.

In Example 20, the subject matter of Example 18 optionally includes a third device having display and configured to receive the first and second brain wave signals and the subsequent brain wave signals from the wearable brain wave sensing device. The wearable device includes an electroencephalogram (EEG) headband, and the second device is a cloud device. The client application is optionally configured to receive the receive the first and second brain wave signals and the subsequent brain wave signals from the third device, and configured to send a message to the third device to present a prompt to the patient on the display regarding a determined therapy state of the patient.

These non-limiting Examples can be combined in any permutation or combination. This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is an illustration of portions of an example of a neurostimulation system.

FIG. 2 is an illustration of portions of another example of a neurostimulation system.

FIG. 3 is an illustration of an example of an implantable pulse generator (IPG) and an implantable lead system.

FIG. 4 is an illustration of another example of an IPG and an implantable lead system.

FIG. 5 is an illustration of an example of an implantable stimulation lead.

FIG. 6 is an illustration of an example of a monitoring system for monitoring brain waves.

FIG. 7 is a flow diagram of an example of a method of monitoring a therapy state of a patient.

FIG. 8 is an illustration of an example of a programming system for a neurostimulation device.

FIG. 9 is a flow diagram of an example of a method of controlling operation of a neurostimulation system.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized, and that structural, logical, and electrical changes may be made without departing from the spirit and scope of the present invention. References to “an”, “one”, or “various” embodiments in this disclosure are not necessarily to the same embodiment, and such references contemplate more than one embodiment. The following detailed description provides examples, and the scope of the present invention is defined by the appended claims and their legal equivalents.

This document discusses devices, systems and methods for programming and delivering electrical neurostimulation to a patient or subject. Advancements in neuroscience and neurostimulation research have led to a demand for delivering complex patterns of neurostimulation energy for various types of therapies. The present system may be implemented using a combination of hardware and software designed to apply any neurostimulation (neuromodulation) therapy, including but not being limited to DBS therapy.

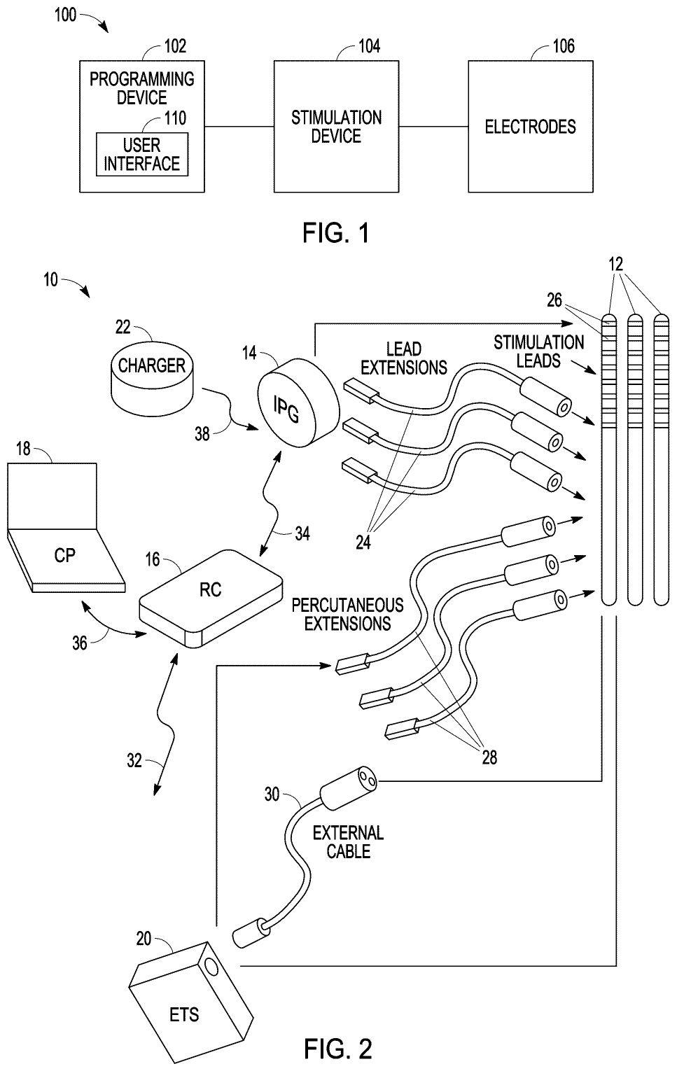

FIG. 1 illustrates an example of portions of a neurostimulation system 100. System 100 includes electrodes 106, a stimulation device 104, and a programming device 102. Electrodes 106 are configured to be placed on or near one or more neural targets in a patient. Stimulation device 104 is configured to be electrically connected to electrodes 106 and deliver neurostimulation energy, such as in the form of electrical pulses, to the one or more neural targets though electrodes 106. The delivery of the neurostimulation is controlled by using multiple stimulation parameters, such as stimulation parameters specifying a pattern of the electrical pulses and a selection of electrodes through which each of the electrical pulses is delivered. In various embodiments, at least some of the stimulation parameters are programmable by a user, such as a physician or other caregiver who treats the patient using system 100. Programming device 102 provides the user with accessibility to the user-programmable parameters. In various embodiments, programming device 102 is configured to be communicatively coupled to stimulation device 104 via a wired or wireless link.

In this document, a “user” includes a physician or other clinician or caregiver who treats the patient using system 100; a “patient” includes a person who receives or is intended to receive neurostimulation delivered using system 100. In various embodiments, the patient can be allowed to adjust his or her treatment using system 100 to certain extent, such as by adjusting certain therapy parameters and entering feedback and clinical effect information.

In various embodiments, programming device 102 can include a user interface 110 that allows the user to control the operation of system 100 and monitor the performance of system 100 as well as conditions of the patient including responses of the patient to the delivery of the neurostimulation. The user can control the operation of system 100 by setting and/or adjusting values of the user-programmable parameters.

In various embodiments, user interface 110 can include a graphical user interface (GUI) that allows the user to set and/or adjust the values of the user-programmable parameters by creating and/or editing graphical representations of various stimulation waveforms. Such waveforms may include, for example, a waveform representing a pattern of neurostimulation pulses to be delivered to the patient as well as individual waveforms that are used as building blocks of the pattern of neurostimulation pulses, such as the waveform of each pulse in the pattern of neurostimulation pulses. The GUI may also allow the user to set and/or adjust stimulation fields each defined by a set of electrodes through which one or more neurostimulation pulses represented by a waveform are delivered to the patient. The stimulation fields may each be further defined by the distribution of the current of each neurostimulation pulse in the waveform. In various embodiments, neurostimulation pulses for a stimulation period (such as the duration of a therapy session) may be delivered to multiple stimulation fields.

In various embodiments, system 100 can be configured for neurostimulation applications. User interface 110 can be configured to allow the user to control the operation of system 100 for neurostimulation. For example, system 100 as well as user interface 110 can be configured for DBS applications. The DBS configurations include various features that may simplify the task of the user in programming the stimulation device 104 for delivering DBS to the patient, such as the features discussed in this document.

FIG. 2 is an illustration of portions of another example of a neurostimulation system 10 that includes one or more stimulation leads 12 and an implantable pulse generator (IPG) 14. The system 10 can also include one or more of an external remote control (RC) 16, a clinician's programmer (CP) 18, an external trial stimulator (ETS) 20, or an external charger 22. The IPG 14 can optionally be physically connected via one or more lead extensions 24, to the stimulation lead(s) 12. Each lead carries multiple electrodes 26 arranged in an array. The IPG 14 includes pulse generation circuitry that delivers electrical stimulation energy in the form of, for example, a pulsed electrical waveform (i.e., a temporal series of electrical pulses) to the electrode array 26 in accordance with a set of stimulation parameters. The IPG 14 can be implanted into a patient's body, for example, below the patient's clavicle area or within the patient's buttocks or abdominal cavity. The implantable pulse generator can have multiple stimulation channels (e.g., 8, 16, or 32) which may be independently programmable to control the magnitude of the current stimulus from each channel. The IPG 14 can have one, two, three, four, or more connector ports, for receiving the terminals of the leads 12.

The ETS 20 may also be physically connected, optionally via the percutaneous lead extensions 28 and external cable 30, to the stimulation leads 12. The ETS 20, which may have similar pulse generation circuitry as the IPG 14, can also deliver electrical stimulation energy in the form of, for example, a pulsed electrical waveform to the electrode array 26 in accordance with a set of stimulation parameters. One difference between the ETS 20 and the IPG 14 is that the ETS 20 is often a non-implantable device that is used on a trial basis after the neurostimulation leads 12 have been implanted and prior to implantation of the IPG 14, to test the responsiveness of the stimulation that is to be provided. Any functions described herein with respect to the IPG 14 can likewise be performed with respect to the ETS 20.

The RC 16 may be used to telemetrically communicate with or control the IPG 14 or ETS 20 via a wireless communications link 32. Once the IPG 14 and neurostimulation leads 12 are implanted, the RC 16 may be used to telemetrically communicate with or control the IPG 14 via communications link 34. The communication or control allows the IPG 14 to be turned on or off and to be programmed with different stimulation parameter sets. The IPG 14 may also be operated to modify the programmed stimulation parameters to actively control the characteristics of the electrical stimulation energy output by the IPG 14. The CP 18 allows a user, such as a clinician, the ability to program stimulation parameters for the IPG 14 and ETS 20 in the operating room and in follow-up sessions. The CP 18 may perform this function by indirectly communicating with the IPG 14 or ETS 20, through the RC 16, via a wireless communications link 36. Alternatively, the CP 18 may directly communicate with the IPG 14 or ETS 20 via a wireless communications link (not shown). The stimulation parameters provided by the CP 18 are also used to program the RC 16, so that the stimulation parameters can be subsequently modified by operation of the RC 16 in a stand-alone mode (i.e., without the assistance of the CP 18).

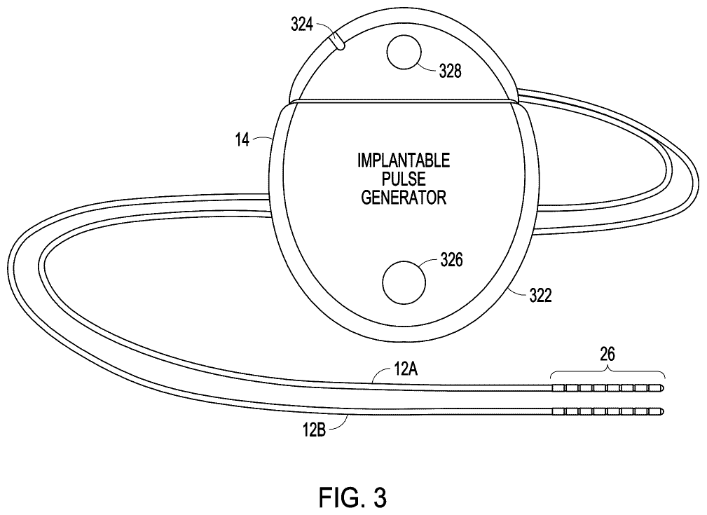

FIG. 3 is an illustration of an example of an IPG 14 (e.g., IPG 14 in FIG. 2) and an implantable lead system that includes stimulation leads (e.g., stimulation leads 12 in FIG. 2). The IPG 14 can be used as stimulation device 104 in FIG. 1. As illustrated in FIG. 3, IPG 14 that can be coupled to implantable leads 12A and 12B at a proximal end of each lead. The distal end of each lead includes electrical contacts or electrodes 26 for contacting a tissue site targeted for electrical neurostimulation. As illustrated in FIG. 3, leads 12A and 12B each include 8 electrodes 26 at the distal end. The number and arrangement of leads 12A and 12B and electrodes 26 as shown in FIGS. 2 and 3 are only examples, and other numbers and arrangements are possible. In various examples, the lead electrodes 26 are ring electrodes. In various examples the lead electrodes 26 include one or more segmented electrodes.

The IPG 14 can include a hermetically sealed IPG case 322 to house the electronic circuitry of IPG 14. IPG 14 can include an electrode 326 formed on IPG case 322. IPG 14 can include an IPG header 324 for coupling the proximal ends of leads 12A and 12B. IPG header 324 may optionally also include an electrode 328. One or both of electrodes 326 and 328 may be used as a reference electrode.

The implantable leads and electrodes may be configured by shape and size to provide electrical neurostimulation energy to a neuronal target included in the subject's brain. Neurostimulation energy can be delivered in a monopolar (also referred to as unipolar) mode using an IPG electrode and one or more electrodes selected from electrodes 26. Neurostimulation energy can be delivered in a bipolar mode using a pair of electrodes of the same lead (lead 12A or lead 12B). Neurostimulation energy can be delivered in an extended bipolar mode using one or more electrodes of a lead (e.g., one or more electrodes of lead 12A) and one or more electrodes of a different lead (e.g., one or more electrodes of lead 12B).

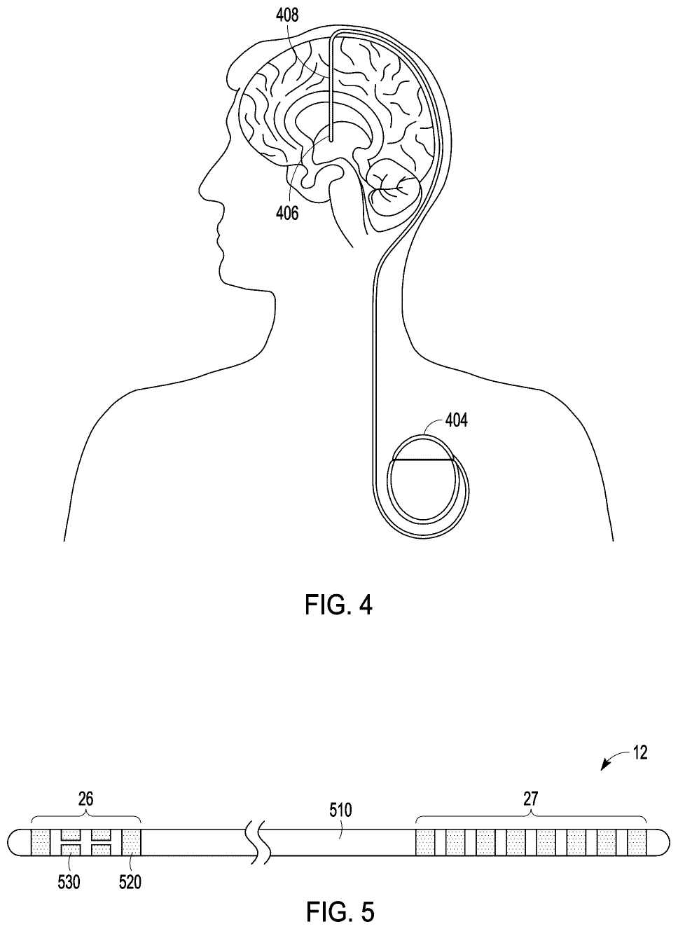

FIG. 4 illustrates another example of an IPG 404 and an implantable lead system 408 arranged to provide neurostimulation to a patient. An example of IPG 404 includes IPG 14 of FIGS. 2 and 3. An example of lead system 408 includes one or more of leads 12A and 12B in FIG. 3. The lead distal end 406 is implanted near a stimulation target. In the illustrated embodiment, implantable lead system 408 is arranged to provide Deep Brain Stimulation (DBS) to a patient, with the stimulation target being neuronal tissue in a subdivision of the thalamus of the patient's brain. Other examples of DBS targets include neuronal tissue of the globus pallidus (GPi), the subthalamic nucleus (STN), the pedunculopontine nucleus (PPN), substantia nigra pars reticulate (SNr), cortex, globus pallidus externus (GPe), medial forebrain bundle (MFB), periaquaductal gray (PAG), periventricular gray (PVG), habenula, subgenual cingulate, ventral intermediate nucleus (VIM), anterior nucleus (AN), other nuclei of the thalamus, zona incerta, ventral capsule, ventral striatum, nucleus accumbens, and any white matter tracts connecting these and other structures.

Returning to FIG. 3, the electronic circuitry of IPG 14 can include a stimulation control circuit that controls delivery of the neurostimulation energy. The stimulation control circuit can include a microprocessor, a digital signal processor, application specific integrated circuit (ASIC), or other type of processor, interpreting or executing instructions included in software or firmware. The neurostimulation energy can be delivered according to specified (e.g., programmed) modulation parameters. Examples of setting modulation parameters can include, among other things, selecting the electrodes or electrode combinations used in the stimulation, configuring an electrode or electrodes as the anode or the cathode for the stimulation, specifying the percentage of the neurostimulation provided by an electrode or electrode combination, and specifying stimulation pulse parameters. Examples of pulse parameters include, among other things, the amplitude of a pulse (specified in current or voltage), pulse duration (e.g., in microseconds), pulse rate (e.g., in pulses per second), and parameters associated with a pulse train or pattern such as burst rate (e.g., an “on” modulation time followed by an “off” modulation time), amplitudes of pulses in the pulse train, polarity of the pulses, etc.

FIG. 5 is a schematic side view of an embodiment of an electrical stimulation lead. FIG. 5 illustrates a stimulation lead 12 with electrodes 26 disposed at least partially about a circumference of the lead 12 along a distal end portion of the lead and terminals 27 disposed along a proximal end portion of the lead. The lead 12 can be implanted near or within the desired portion of the body to be stimulated (e.g., the brain, spinal cord, or other body organs or tissues). In one example of operation for deep brain stimulation, access to the desired position in the brain can be accomplished by drilling a hole in the patient's skull or cranium with a cranial drill (commonly referred to as a burr), and coagulating and incising the dura mater, or brain covering. The lead 12 can be inserted into the cranium and brain tissue with the assistance of a stylet (not shown). The lead 12 can be guided to the target location within the brain using, for example, a stereotactic frame and a microdrive motor system. In some embodiments, the microdrive motor system can be fully or partially automatic. The microdrive motor system may be configured to perform one or more the following actions (alone or in combination): insert the lead 12, advance the lead 12, retract the lead 12, or rotate the lead 12.

The lead 12 for deep brain stimulation can include stimulation electrodes. In at least some embodiments, the lead 12 is rotatable so that the stimulation electrodes can be aligned with the target neurons after the neurons have been located using the recording electrodes. Stimulation electrodes may be disposed on the circumference of the lead 12 to stimulate the target neurons. Stimulation electrodes may be ring-shaped so that current projects from each electrode equally in every direction from the position of the electrode along a length of the lead 12. In the embodiment of FIG. 5, two of the electrodes 520 are ring electrodes 520. Ring electrodes typically do not enable stimulus current to be directed from only a limited angular range around of the lead. Segmented electrodes 530, however, can be used to direct stimulus current to a selected angular range around the lead. When segmented electrodes 530 are used in conjunction with an IPG 14 that delivers constant current stimulus, current steering can be achieved to more precisely deliver the stimulus to a position around an axis of the lead (e.g., radial positioning around the axis of the lead). To achieve current steering, segmented electrodes can be utilized in addition to, or as an alternative to, ring electrodes.

The lead 12 includes a lead body 510, terminals 27, and one or more ring electrodes 520 and one or more sets of segmented electrodes 530 (or any other combination of electrodes). The lead body 510 can be formed of a biocompatible, non-conducting material such as, for example, a polymeric material. Suitable polymeric materials include, but are not limited to, silicone, polyurethane, polyurea, polyurethaneurea, polyethylene, or the like. Once implanted in the body, the lead 12 may be in contact with body tissue for extended periods of time. In at least some embodiments, the lead 12 has a cross-sectional diameter of no more than 1.5 millimeters (1.5 mm) and may be in the range of 0.5 to 1.5 mm. In at least some embodiments, the lead 12 has a length of at least 10 centimeters (10 cm) and the length of the lead 12 may be in the range of 10 to 70 cm.

The electrodes 26 can be made using a metal, alloy, conductive oxide, or any other suitable conductive biocompatible material. Examples of suitable materials include, but are not limited to, platinum, platinum iridium alloy, iridium, titanium, tungsten, palladium, palladium rhodium, or the like. Preferably, the electrodes are made of a material that is biocompatible and does not substantially corrode under expected operating conditions in the operating environment for the expected duration of use. Each of the electrodes can either be used or unused (OFF). When the electrode is used, the electrode can be used as an anode or cathode and carry anodic or cathodic current. In some instances, an electrode might be an anode for a period of time and a cathode for a period of time.

Deep brain stimulation leads and other leads may include one or more sets of segmented electrodes. Segmented electrodes may provide for superior current steering than ring electrodes because target structures in deep brain stimulation or other stimulation are not typically symmetric about the axis of the distal electrode array. Instead, a target may be located on one side of a plane running through the axis of the lead. Through the use of a radially segmented electrode array (“RSEA”), current steering can be performed not only along a length of the lead but also around a circumference of the lead. This provides precise three-dimensional targeting and delivery of the current stimulus to neural target tissue, while potentially avoiding stimulation of other tissue.

Any number of segmented electrodes 530 may be disposed on the lead body 510 including, for example, anywhere from one to sixteen or more segmented electrodes 530. It will be understood that any number of segmented electrodes 530 may be disposed along the length of the lead body 510. A segmented electrode 530 typically extends only 75%, 67%, 60%, 50%, 40%, 33%, 25%, 20%, 17%, 15%, or less around the circumference of the lead.

The segmented electrodes 530 may be grouped into sets of segmented electrodes, where each set is disposed around a circumference of the lead 12 at a particular longitudinal portion of the lead 12. The lead 12 may have any number segmented electrodes 530 in a given set of segmented electrodes. The lead 12 may have one, two, three, four, five, six, seven, eight, or more segmented electrodes 530 in a given set. In at least some embodiments, each set of segmented electrodes 530 of the lead 12 contains the same number of segmented electrodes 530. The segmented electrodes 530 disposed on the lead 12 may include a different number of electrodes than at least one other set of segmented electrodes 530 disposed on the lead 12. The segmented electrodes 530 may vary in size and shape. In some embodiments, the segmented electrodes 530 are all of the same size, shape, diameter, width or area or any combination thereof. In some embodiments, the segmented electrodes 530 of each circumferential set (or even all segmented electrodes disposed on the lead 12) may be identical in size and shape.

Each set of segmented electrodes 530 may be disposed around the circumference of the lead body 510 to form a substantially cylindrical shape around the lead body 510. The spacing between individual electrodes of a given set of the segmented electrodes may be the same, or different from, the spacing between individual electrodes of another set of segmented electrodes on the lead 12. In at least some embodiments, equal spaces, gaps or cutouts are disposed between each segmented electrode 530 around the circumference of the lead body 510. In other embodiments, the spaces, gaps or cutouts between the segmented electrodes 530 may differ in size, or cutouts between segmented electrodes 530 may be uniform for a particular set of the segmented electrodes 530 or for all sets of the segmented electrodes 530. The sets of segmented electrodes 530 may be positioned in irregular or regular intervals along a length the lead body 510.

Conductor wires (not shown) that attach to the ring electrodes 520 or segmented electrodes 530 extend along the lead body 510. These conductor wires may extend through the material of the lead 12 or along one or more lumens defined by the lead 12, or both. The conductor wires couple the electrodes 520, 530 to the terminals 27.

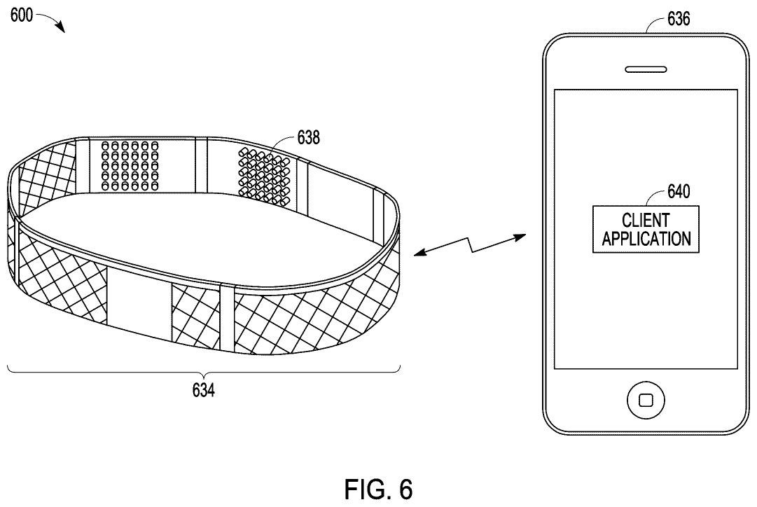

FIG. 6 illustrates an example of a monitoring system 600 for monitoring brain waves of a patient. The monitoring system 600 includes a wearable brain wave signal sensing device 634 and a second device 636 that communicates information wirelessly with the wearable device. The second device 636 may be a personal device in possession of the patient. In the example of FIG. 6, the wearable brain wave signal sensing device 634 is an electroencephalogram (EEG) sensing headband and the second device 636 is a smartphone. The EEG headband includes electrodes 638 that sense brain wave signals of the wearer. The EEG headband can digitize the sensed waveforms and send brain wave information to the smartphone. The smartphone includes a client application 640 that executes on the processing circuitry of the smartphone. The client application 640 can receive the brain wave signals from the EEG headband. The client application 640 may also be able to receive input from the patient regarding their evaluation of their therapy state. The monitoring system 600 is useful to determine the medication state of the patient.

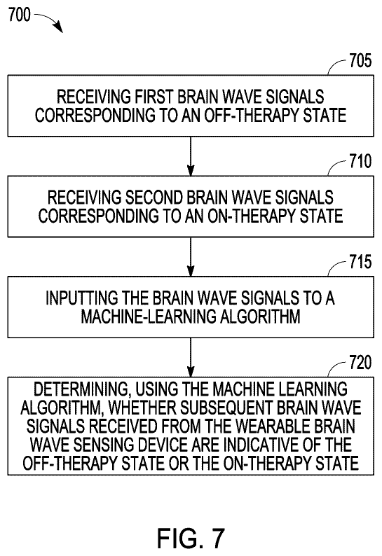

FIG. 7 is a flow diagram of a method 700 of monitoring a medication state of a patient. The patient may be prescribed drug medication for their disease (e.g., Parkinson's disease). The method 700 provides technique of detecting when the patient is following their medication regimen. The method 700 can be performed using the monitoring system 600 of FIG. 6. The method 700 may be optimized in a clinical setting and then performed while the patient is ambulatory and outside of a clinical setting.

At block 705, first brain wave signals are received by the client application 640 from the wearable brain wave signal sensing device 634. The first set of brain wave signals correspond to an off-therapy state of the patient (e.g., off-medication state, off-stimulation state, etc.). The patient may be given a benchmark test to assess the off-therapy state of the patient while the patient is off their therapy. Brain wave signals are monitored and recorded by the wearable brain wave signal sensing device 634 while it is worn by the patient.

At block 710, second brain wave signals are received by the client application 640 from the wearable brain wave signal sensing device 634. The second set of brain wave signals correspond to an on-therapy state of the patient (e.g., on-medication state, on-stimulation state, etc.). corresponding to an on-therapy state of the patient. The patient may be given the same benchmark test to assess the on-therapy state of the patient while the patient is on medication. Brain wave signals are again monitored and recorded by the wearable brain wave signal sensing device 634 while it is worn by the patient.

At block 715, the first and second set of brain wave signals are input to a machine learning algorithm performed by the second device 636. The recorded off-therapy brain wave signals and the recorded on-therapy brain wave signals are training data for the machine learning algorithm to learn to automatically distinguish between sensed brain wave signals corresponding to the off-therapy state and sensed brain wave signals corresponding to the on-therapy state.

At block 720, using the machine learning algorithm, the client application 640 determines whether subsequent brain wave signals received from the wearable brain wave signal sensing device are indicative of the off-therapy state of the patient or the on-therapy state of the patient. This allows the client application 640 to determine the therapy state of the patient. If the client application 640 determines from the brain wave signals that the patient is in an off-therapy state, the client application 640 may begin presenting prompts to the patient to resume the therapy (e.g., to resume taking their medication or enabling stimulation). The prompts can include a displayed prompt, text message prompt or audible prompt from the second device 636. If the second device 636 is a cloud device, the cloud device may send a message or command to a personal device of the patient or user to present a prompt to the user or patient. Also, these subsequently received brainwaves may be matched with the patient's feedback about their therapy state to further train the machine learning algorithm to improve the classification performance of off-therapy and on-therapy brain signals.

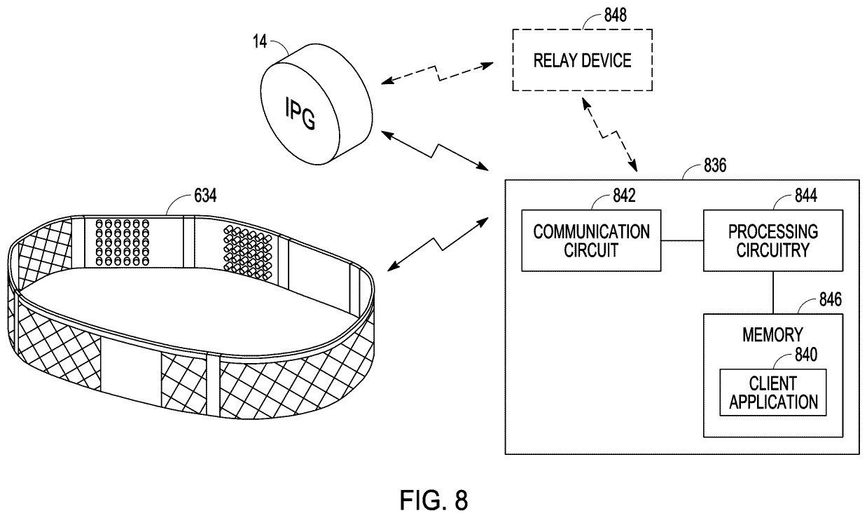

FIG. 8 illustrates a programming system for a neurostimulation device used by the patient. The neurostimulation device may be an IPG 14 that provides neurostimulation energy to a patient according to programmable stimulation parameters. The neurostimulation device includes implantable stimulation leads (not shown) that include electrodes to deliver the neurostimulation energy to tissue of the patient. The programming system includes a wearable brain wave signal sensing device 634 and a second device 836 that communicates information wirelessly with the wearable device and the neurostimulation device.

The wearable brain wave signal sensing device 634 senses brain wave signals of a patient. The brain wave signals may be produced in response to neurostimulation delivered using the neurostimulation device, and the wearable device may sense the brain wave signals in a time relationship to the delivery of the neurostimulation. The second device 836 includes a communication circuit 842, processing circuitry 844, and a client application 840. The communication circuit 842 communicates information wirelessly with the wearable brain wave signal sensing device 634 and the neurostimulation device. The processing circuitry 844 may include one or more processors (e.g., a microprocessor, a digital signal processor, application specific integrated circuit (ASIC), or other type of processor) interpreting or executing instructions included in software or firmware. The instructions performed by the processing circuitry 844 (e.g., instructions included in the client application 840) may be stored in a memory 846 separate from or integral to the processing circuitry 844.

The second device 836 communicates stimulation parameters to the neurostimulation device, and the neurostimulation device delivers neurostimulation energy according to the specified stimulation parameters. Examples of setting stimulation parameters can include, among other things, selecting the electrodes or electrode combinations used in the stimulation, configuring an electrode or electrodes as the anode or the cathode for the stimulation, specifying the percentage of the neurostimulation provided by an electrode or electrode combination, and specifying stimulation pulse parameters. Examples of pulse parameters include, among other things, the amplitude of a pulse (specified in current or voltage), pulse duration (e.g., in microseconds), pulse rate (e.g., in pulses per second), and parameters associated with a pulse train or pattern such as burst rate (e.g., an “on” modulation time followed by an “off” modulation time), amplitudes of pulses in the pulse train, polarity of the pulses, etc.

The stimulation lead that delivers the neurostimulation energy includes multiple electrodes and the electrodes can be configured into multiple electrode configurations by the second device 836. The stimulation parameters can include different electrode configurations to steer the neurostimulation energy (e.g., electrical current) toward different volumes of tissue. Additionally, the electrode configuration can include “fractionalization” of the electrical current flowing through the selected one or more electrodes. In fractionalization, a fraction of an overall pulse amplitude is assigned to each of the electrodes included in the electrode configuration.

In some examples, the wearable brain wave signal sensing device 634 is an EEG sensing headband and the second device 836 is a device personal to the patient such as a smartphone with a programming App for the neurostimulation device. In some examples, the second device 836 is a clinical programmer (e.g., CP 18 in FIG. 1). In some examples, the second device 836 is a cloud device (e.g., a cloud server) located in the cloud. The programming system may include a relay device 848 that serves as an intermediate communication device in the communication between any combination of the wearable device, the neurostimulation device, and the cloud device. The term “cloud” is used herein to refer to a hardware abstraction. Instead of one dedicated server processing uploaded brain wave signal information, uploading the brain wave information to the cloud can include the relay device 848 sending brain signal information to a data center or processing center (e.g., using the internet). The actual server used to process the information may be interchangeable at the data center or processing center.

The brain wave signals sensed by the wearable device provide feedback for the neurostimulation energy delivered according to the stimulation regimen programmed by the second device 836. The client application 840 uses the brain wave signal information to determine if the neurostimulation energy produced the desired target response in the patient indicating the desired therapy state was produced. Although the neurostimulation is delivered using implantable leads of the neurostimulation device, the response brain wave signals are sensed using the wearable device. This eliminates the challenges of trying to sense a response to neurostimulation using electrodes of the same lead used to deliver the neurostimulation energy.

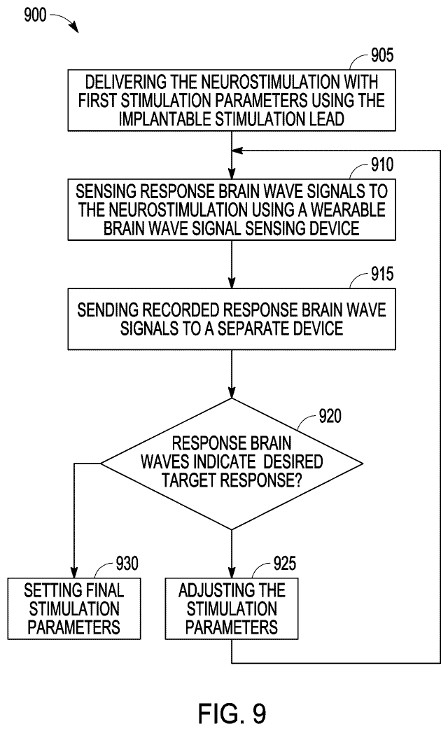

FIG. 9 is a flow diagram of an example of a method 900 of controlling operation of a neurostimulation system. The neurostimulation system may include the neurostimulation device and programming system of FIG. 8. The neurostimulation device includes one or more implantable stimulation leads with electrodes to deliver the neurostimulation energy to the patient.

At block 905, the neurostimulation device (e.g., IPG 14 in FIG. 8) delivers neurostimulation using the implantable lead. The neurostimulation is delivered with first stimulation parameters. The first parameters may include, among other things, a first electrode fractionalization and first stimulation amplitude. The neurostimulation may cause a response in brain neurons of the patient that may be evident in EEG signals.

At block 910, response brain wave signals to the neurostimulation are sensed using the wearable brain wave signal sensing device 634. The brain wave signals may be sensed and recorded in a specified time relationship to the neurostimulation.

At block 915, the response brain wave signal information is sent to the second device 836 and received by the client application 840 for signal processing. At block 920, the client application 840 determines the sensed response of the patient to the neurostimulation using the signal processing of the received brain wave signal information. The sensed brain wave signals provide feedback regarding whether the programmed neurostimulation produced the desired target response in the patient.

In some examples, the client application 840 compares response brain wave signals to a stored target response stored in the second device 836. The target response may include a target brain wave signal (e.g., an EEG signature), and the client application 840 determines that the sensed brain wave signals show the desired target response was produced when the sensed brain wave signals correlate well with the target brain wave signal. In some examples, the desired target response is a target frequency response in brain signals. The client application 840 determines that the sensed brain wave signals show the desired target response was produced when the sensed brain wave signals include the target frequency response.

To determine that the sensed response brain wave signals correlate well to the target response, the sensed signals may be analyzed using signal-processing and feature extraction techniques. The feature extraction techniques identify signal features from the sensed signals and perform metrics on the features to determine if the features correlate to the desired target response.

In some examples, the desired target response may be a predetermined sequence of brain wave signals over time. The client application 840 determines that the sensed brain wave signals show desired therapy response was produced when the sensed brain wave signals correlate well with the sequence of brain wave signals. In some examples, the sensed brain wave signals correspond to a neuron rest state after a neuron stimulation state produced by the neurostimulation. The client application 840 determines that the sensed brain wave signals show the desired target response was produced when the brain waves indicate the target rest state is produced.

If the second device 836 is a smartphone the client application (or smartphone App) may prompt the user for information regarding the neurostimulation therapy. For instance, the prompt may include questions about the satisfaction of the patient with the neurostimulation, if the neurostimulation caused a particular side effect, did the neurostimulation relieve a symptom, etc. The client application 840 may determine the sensed response of the patient to the neurostimulation using the sensed brain wave signals and the feedback information from the patient or user. The patient feedback information can be used to train the machine learning algorithm to identify which are the brain waves that most likely represent a good therapy state.

At block 925, the second device 836 adjusts the stimulation parameters of the neurostimulation device if the comparison of the sensed response and the desired target response indicates that the desired target response was not produced. For instance, the second device 836 may recurrently program different fractionalizations into the neurostimulation device and deliver the reprogrammed stimulation to see if it produces the desired target response. The second device 836 may also recurrently program different pulse amplitudes and deliver the reprogrammed stimulation to see if a stimulation configuration produces the desired target response.

The second device 836 may define a pattern of neurostimulation pulses for delivery based on the feedback brain wave signals by creating or adjusting one or more stimulation waveforms. The pattern definition can also include definition of one or more stimulation fields each associated with one or more pulses in the pattern of neurostimulation pulses. A stimulation configuration can include the pattern of neurostimulation pulses including the one or more stimulation fields, or at least various aspects or parameters of the pattern of neurostimulation pulses including the one or more stimulation fields. Stimulation configuration can include the electrode configuration used to provide the electrical stimulation. The neurostimulation programming by the second device 836 can include the definition of the one or more stimulation waveforms, including the definition of one or more stimulation fields.

There may be many combinations of simulation parameters and the number of programmable options available can become large. This may produce a large parameter space. The client application 840 may input one or more of the sensed brain wave signals and the stimulation parameters that produced the sensed brain wave signals into a machine learning algorithm performed by the second device 836. The output of the machine learning algorithm may be a combination of stimulation parameters that are programmed into the neurostimulation device. The machine learning algorithm may reduce the number of options to try for the stimulation programming based on the current sensed brain wave signals and the current stimulation parameters. The machine learning algorithm may output stimulation parameters to reduce a difference between the desired target response brain wave signal and the sensed brain wave signals and the client application programs the adjusted parameters into the neurostimulation device.

At block 930, if the response brain wave signals from the wearable device indicate that the stimulation produced the desired target response the stimulation parameters that produced the response are set as the final parameters of the testing. Thus, the client application 840 customizes the stimulation parameters of the patient to achieve the desired target response.

The devices, systems and methods described herein provide techniques to address the challenges in stimulation lead based sensing regarding low signal to noise ratio, heart activity, movement artifacts, ambiguity of the electrode recording the neuron activity and inability to use some stimulation-based features of the neurostimulation system die to constraints of lead-based signal recording. The techniques also address the challenges due to the high amount of energy used by the implantable device when using the implantable device for sensing and recording.

The embodiments described herein can be methods that are machine or computer-implemented at least in part. Some embodiments may include a computer-readable medium or machine-readable medium encoded with instructions operable to configure an electronic device or system to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times. Various embodiments are illustrated in the figures above. One or more features from one or more of these embodiments may be combined to form other embodiments.

The above detailed description is intended to be illustrative, and not restrictive. The scope of the disclosure should, therefore, be determined with references to the appended claims, along with the full scope of equivalents to which such claims are entitled.

What is claimed is:

1. A programming system for a neurostimulation device that provides neurostimulation energy to a patient according to programmable stimulation parameters, the programming system comprising:
a wearable brain wave signal sensing device configured to sense brain wave signals of a patient produced in response to neurostimulation delivered using the neurostimulation device; and
a second device including:
a communication circuit to receive brain wave signal information from the wearable brain wave signal sensing device;
processing circuitry and a client application including instructions executable by the processing circuitry and configured to:
determine a sensed response to the neurostimulation of the patient indicated by the received brain wave signal information;
compare the sensed response to a desired target response to the neurostimulation; and
adjust the stimulation parameters of the neurostimulation device according to the comparing of the sensed response and the desired target response.

2. The programming system of claim 1,
wherein the wearable brain wave sensing device includes an electroencephalogram (EEG) headband; and
wherein the second device is a smartphone and the client application is configured to:
receive the sensed brain wave signals produced by the EEG headband;
perform signal processing on the sensed brain wave signals to determine whether the sensed brain wave signals correspond to the desired target response to the neurostimulation; and
program the neurostimulation device with the adjusted stimulation parameters.

3. The programming system of claim 2, wherein the client application is configured to:
receive information from a user of the smartphone regarding the neurostimulation delivered using the neurostimulation device; and
determine the sensed response of the patient to the neurostimulation using the sensed brain wave signals and the information from the user.

4. The programming system of claim 1,
wherein the wearable brain wave sensing device includes an electroencephalogram (EEG) headband, and the wearable brain wave sensing device is configured to send the sensed brain wave signals to a personal device of the patient; and
wherein the second device is a cloud device and the client application is configured to:
receive the sensed brain wave signals from the personal device;
perform signal processing on the sensed brain wave signals to determine the sensed response to the neurostimulation indicated by the sensed brain waves and compare the sensed response to the desired target response to the neurostimulation; and
send the adjusted stimulation parameters to the personal device.

5. The programming system of claim 1,
wherein the wearable brain wave sensing device includes an electroencephalogram (EEG) headband; and
wherein the client application is configured to:
compare the sensed brain wave signals sensed with the EEG headband to a target response brain wave signal; and
adjust the stimulation parameters of the neurostimulation device to change the sensed brain wave signals to reduce a difference between the target response brain wave signal and the sensed brain wave signals sensed with the EEG headband.

6. The programming system of claim 1, wherein the client application is configured to:
set the stimulation parameters of the neurostimulation device to include a first stimulation energy amplitude and a first electrode fractionalization;
determine whether the sensed brain wave signals produced by the wearable brain wave sensing device indicate that the first stimulation energy amplitude and a first electrode fractionalization produce the desired target response to the neurostimulation; and
change one or both of the electrode fractionalization and the stimulation energy amplitude when the sensed brain wave signals indicate that the first stimulation parameters did not produce the desired target response.

7. The programming system of claim 6, wherein the client application is configured to:
determine a frequency response of the sensed brain wave signals sensed using the wearable brain wave sensing device;
compare the determined frequency response to a target frequency response; and
adjust the stimulation parameters to change the frequency response of the sensed brain wave signals to reduce a difference between the determined frequency and the target frequency response.

8. A method of controlling operation of a neurostimulation system to deliver electrical neurostimulation to tissue of a patient using an implantable stimulation lead that includes electrodes, the method comprising:
delivering the neurostimulation with first stimulation parameters using the implantable stimulation lead;
sensing response brain wave signals to the neurostimulation using a wearable brain wave signal sensing device;
sending recorded response brain wave signals to a separate device;
determining, using the separate device, a response of the patient to the neurostimulation indicated by the recorded response brain wave signals; and
recurrently adjusting the stimulation parameters to adjust the response to the neurostimulation indicated by the recorded response brain waves.

9. The method of claim 8,
wherein the sending the recorded response brain wave signals includes:
sending first brain wave signals from the wearable brain wave signal sensing device corresponding to an off-therapy state of the patient; and
sending second brain wave signals from the wearable brain wave signal sensing device corresponding to an on-therapy state of the patient; and
wherein the determining the neurostimulation response includes:
inputting the first and second brain wave signals to a machine learning algorithm of the separate device; and
determining, using the machine learning algorithm, whether subsequent brain wave signals sent from the wearable brain wave signal sensing device are indicative of the response to the neurostimulation being the off-therapy state of the patient or the on-therapy state of the patient.

10. The method of claim 8,
wherein the sensing the response brain wave signals includes sensing the brain wave signals using an electroencephalogram (EEG) headband; and
wherein the sending the recorded response brain waves includes sending the recorded response brain waves to a personal device of the patient that performs signal processing on the response brain waves to determine whether the response brain waves correspond to a desired target response to the neurostimulation.

11. The method of claim 10,
wherein the recurrently adjusting the stimulation parameters includes recurrently changing the stimulation parameters using the personal device of the patient.

12. The method of claim 11,
wherein the delivering the neurostimulation includes delivering the neurostimulation using an implantable pulse generator; and
wherein the personal device of the patient is a smartphone that includes an application to change the stimulation parameters.

13. The method of claim 8,
wherein the sensing using the wearable brain wave sensing device includes sensing the brain waves using an electroencephalogram (EEG) headband; and
wherein the sending the recorded response brain waves includes sending the recorded response brain waves to a cloud device that performs signal processing on the response brain waves to determine whether the response brain waves correspond to a desired target response to the neurostimulation.

14. The method of claim 8,
wherein the delivering the neurostimulation with the first stimulation parameters includes delivering the neurostimulation with a first stimulation energy amplitude and a first electrode fractionalization of the stimulation lead;
wherein the determining the neurostimulation response includes determining whether the recorded response brain wave signals indicate that the first stimulation parameters produce a desired target response to the neurostimulation energy; and
wherein the recurrently adjusting the stimulation parameters includes changing one or both of the electrode fractionalization and the stimulation energy amplitude when the recorded response brain wave signals indicate that the first stimulation parameters did not produce the desired target response.

15. The method of claim 8, including:
comparing, by the separate device, the recorded response brain wave signals to a predetermined sequence of brain wave signals; and
wherein the recurrently adjusting the stimulation parameters includes recurrently adjusting the stimulation parameters to reduce a difference between the sensed response brain wave signals and the predetermined sequence of brain wave signals.

16. The method of claim 8, wherein the recurrently adjusting the stimulation parameters includes:
comparing, by the separate device, the recorded response brain wave signals to a target response brain wave signal; and
adjusting the stimulation parameters to change the sensed response brain wave signals to reduce a difference between the sensed response brain wave signals and the target response brain wave signal.

17. The method of claim 8,
wherein the sensing using the wearable brain wave sensing device includes sensing the brain wave signals using an electroencephalogram (EEG) headband;
wherein the determining the neurostimulation response includes determining, using the separate device, a frequency response of the sensed brain wave signals sensed using the EEG headband; and
wherein the recurrently adjusting the stimulation parameters includes:
comparing, by the separate device, the determined frequency response to a target frequency response; and
adjusting the stimulation parameters to change the frequency response of the sensed brain wave signals to reduce a difference between the frequency response of the recorded signals and the target frequency response.

18. A monitoring system comprising:
a wearable brain wave sensing device configured to sense brain wave signals of a patient; and
a second device including a client application configured to:
receive first brain wave signals from the wearable brain wave sensing device corresponding to an off-therapy state of the patient;
receive second brain wave signals from the wearable brain wave sensing device corresponding to an on-therapy state of the patient;
input the first and second brain wave signals to a machine learning algorithm; and
determine, using the machine learning algorithm, whether subsequent brain wave signals received from the wearable brain wave sensing device are indicative of the off-medication state of the patient or the on-therapy state of the patient.

19. The monitoring system of claim 18,
wherein the wearable brain wave sensing device includes an electroencephalogram (EEG) headband;
wherein the second device is a smartphone and the client application is an application (App) performable by processing circuitry of the smartphone; and
wherein the App is configured to present a prompt on a display of the smartphone regarding a determined therapy state of the patient.

20. The monitoring system of claim 18, including:
including a third device having display and configured to receive the first and second brain wave signals and the subsequent brain wave signals from the wearable brain wave sensing device;
wherein the wearable brain wave sensing device includes an electroencephalogram (EEG) headband; and
wherein the second device is a cloud device and the client application is configured to receive the receive the first and second brain wave signals and the subsequent brain wave signals from the third device, and configured to send a message to the third device to present a prompt to the patient on the display regarding a determined therapy state of the patient.
